(19) United States
(12) Patent Application Publication
(10) Pub. No.: US 2021/0386484 A1
(43) Pub. Date: Dec. 16, 2021

(54) Dental Tool System

(71) Applicant: Ivoclar Vivadent AG, Schaan (LI)

(72) Inventors: Stéfane Follonier, Vilters (CH); Wolfgang Wachter, Schaan (LI)

(21) Appl. No.: 17/281,629

(22) PCT Filed: Sep. 12, 2019
(86) PCT No.: PCT/EP2019/074331

§ 371 Date: Mar. 31, 2021

(30) Foreign Application Priority Data
Oct. 9, 2018 (EP) 18199460.9

(51) Int. Cl. A61B 34/20; A61B 1/24; A61B 1/00; H04N 13/239; G06T 7/00; G06T 7/70; H04N 5/225; G06K 9/00; G02B 27/01; G16H 30/40

(52) U.S. Cl. CPC A61B 34/20; A61B 1/24; A61B 1/00048; A61B 1/00193; H04N 13/239; G06T 7/0012; A61B 2090/502; H04N 5/2257; G06K 9/00664; G02B 27/0172; G16H 30/40; A61B 2034/2055; G06T 7/70

(57) ABSTRACT

The present invention relates to a dental tool system, comprising a detection system (101) for detecting a spatial position (103-Z) of a tooth (115) or a tooth situation and a spatial tool position (103-W) of a tool (107); and a device (105) for controlling or regulating the tool (107) on the basis of the detected tool position (103-W) in relation to the position (103-Z) of the tooth (115) or the tooth situation.

The present invention relates to a dental tool system and a method for controlling a dental tool.

Dental tools for dental treatment, such as drills or polymerisation lamps, can be operated incorrectly. This can result in irreparable harm to a patient during a dental treatment.

Therefore, it is the technical object of the present invention to technically simplify handling of controllable dental tools.

This object is achieved by the subject-matter according to the independent claims. Advantageous embodiments are subject to the dependent claims, the description and the figures.

According to a first aspect, the present object is achieved by means of a dental tool system, comprising a detection system for detecting a spatial position of a tooth or a tooth situation and a spatial tool position of a tool; and a device for controlling or regulating the tool on the basis of the detected tool position in relation to the position of the tooth or the detected tooth situation. The detection of the position of the tooth or the tooth situation can be performed by means of an optical method or an X-ray method. In general, the electronic detection system can be any detection system which can be used to obtain a position of the tooth or the tooth situation.

Controlling or regulating can be affected by calculating a spatial distance between the tool position and the position of the tooth or the tooth situation. Controlling or regulating on the basis of the detected tool position in relation to the position of the tooth or the tooth situation can be effected e.g. if the tool position is outside a specified spatial region with respect to the position of the tooth or the tooth situation or if the distance between the tool position and the position of the tooth or the tooth situation exceeds or is less than a specified distance. The tooth used to detect the position of the tooth or the tooth situation can be selected via a user interface. Detecting the position of the tooth or the tooth situation and the tool position can be performed continuously in real time. The dental tool system provides e.g. the technical advantage that a correct use of the dental tool in relation to the tooth to be treated or the location to be treated can be ensured and a position-based control of the tool can be performed.

The tooth situation is e.g. a situation of the tooth or the tooth root which is determined by the shape, the interior or a caries infestation. In general, the tooth situation can be given by each spatial region of the tooth requiring processing by the tool.

For example, a drill as a tool can be switched-on on the basis of the position of the tooth situation as long as it treats a carious region of the tooth. The information relating to the tooth situation can be obtained e.g. using an X-ray method. The depth information or X-ray images can indicate the position of the caries infestation in the tooth.

In a technically advantageous embodiment of the dental tool system, an optical recording device of the electronic detection system is integrated into data glasses. The data glasses are an electronic apparatus which are worn as glasses and can be used to optically display further information to a user in addition to the natural visual perception. By means of a display device, the data glasses can superimpose additional information on an image which is perceived by the wearer's eye. The display device comprises e.g. a display screen close to the eyes or a projector for directly projecting onto the retina. By integrating the detection system into the data glasses, e.g. the technical advantage is achieved that the structure of the dental guide system is simplified.

In a further technically advantageous embodiment of the dental tool system, the dental tool system is designed to display an error visually on a display device of the data glasses. The error is e.g. an error on the basis of the detected tool position in relation to the position of the tooth or the tooth situation. The error is produced e.g. if the tool exceeds or is less than a specific distance from the tooth. This provides e.g. the technical advantage that the user can immediately recognise an incorrect position of the tool.

In a further technically advantageous embodiment of the dental tool system, the detection system comprises a stereoscopic detection system having a first and a second camera. This provides e.g. the technical advantage that the positions can be detected with small technical effort and the detection system can be easily integrated into data glasses.

In a further technically advantageous embodiment of the dental tool system, the detection system comprises a camera for detecting the position of the tooth or the tooth situation and the tool position based on a travel time measurement of light. This provides e.g. the technical advantage that the positions can be detected quickly and with a high level of accuracy.

In a further technically advantageous embodiment of the dental tool system, the detection system comprises a projection device for projecting a light pattern. The detection system can detect the projected light pattern. The spatial position of the tooth or the tooth situation can be calculated based on the detected light pattern. This provides e.g. the technical advantage that accurate position detection can be achieved.

In a further technically advantageous embodiment of the dental tool system, the detection system is designed to detect the position of the tooth or the tooth situation and the tool position on the basis of a time sequence of images. This provides e.g. the technical advantage that the positions of the tooth or the tooth situation and the tool position can be detected easily and accurately. Each further image of the sequence increases the accuracy of the detected position of the tooth or the tooth situation and tool position. For this purpose, specific computer algorithms can be used.

In a further technically advantageous embodiment of the dental tool system, the electronic detection system is designed to determine a time interval in which the tool position is within a specified spatial region with respect to the position of the tooth or the tooth situation. This provides e.g. the technical advantage that time-control of the tool can be performed, e.g. the tool can be switched off after a specified time interval within a specified spatial region has elapsed.

In a further technically advantageous embodiment of the dental tool system, the electronic detection system is designed to optically or electronically recognise the type of the used tool.

The tool is optically recognised e.g. by a comparison of a recorded image of the tool with a previously stored image of the tool. The tool is electronically recognised e.g. wirelessly by means of electromagnetic waves. For this purpose, the tool can comprise a transmitter-receiver system for automatically and contactless identifying the tool with radio waves. For example, an RFID chip is disposed in the tool that identifies the tool and is being read out by a receiver. This provides e.g. the technical advantage that suitable control can be selected automatically on the basis of the recognised tool.

In a further technically advantageous embodiment of the dental tool system, the tool comprises a polymerisation lamp, a dental drill, an endoscope, a camera, a scanner, a mirror, a light-curing apparatus, a dentist's chair, a light source for diagnostic purposes or a light source for colour determination. This provides e.g. the technical advantage that particularly suitable tools are used for control purposes.

In a further technically advantageous embodiment of the dental tool system, the detection system comprises an autofocus function or zoom function for detecting the position of the tooth or the tooth situation and/or tool position. The autofocus function permits automatic focussing by adapting a camera setting to the distance between the camera and tooth or tool and by imaging the tooth or tool in a sharp manner. The zoom function allows the image section to be continuously adapted to the tooth or the tool. This provides e.g. the technical advantage that the operation and handling of the dental tool system are simplified.

In a further technically advantageous embodiment of the dental tool system, the detection system is designed to detect the spatial position of the tooth or the tooth situation based on a sequence of tooth images which have been obtained from different viewing angles. This provides e.g. the technical advantage that simple and rapid position detection can be achieved.

According to a second aspect, the object is achieved by means of a method for controlling a dental tool, comprising the steps of detecting a spatial position of a tooth or a tooth situation and a spatial tool position of a tool; and controlling or regulating the tool on the basis of the detected tool position in relation to the position of the tooth or the tooth situation. Controlling or regulating on the basis of the detected tool position in relation to the position of the tooth or the tooth situation can be affected e.g. if the tool position is outside a specified spatial region with respect to the position of the tooth or the tooth situation. The method provides the same technical advantages as the dental tool system according to the first aspect.

In a technically advantageous embodiment of the method, an error is optically displayed on a display device of data glasses. This likewise provides e.g. the technical advantage that the design of the dental tool system is simplified.

In a further technically advantageous embodiment of the method, the position of the tooth or the tooth situation and the tool position are detected by a stereoscopic detection system or by a detection system based on a travel time measurement of light. This likewise provides e.g. the technical advantage that the positions can be detected quickly and with a high level of accuracy.

In a further technically advantageous embodiment of the method, the position of the tooth or the tooth situation and the tool position are detected on the basis of a sequence of images. This likewise provides e.g. the technical advantage that the position of the tooth or the tooth situation and the tool position can be detected easily and accurately.

In a further technically advantageous embodiment of the method, a time interval is determined in which the tool position is within a specified spatial region with respect to the position of the tooth or the tooth situation. This provides e.g. the technical advantage that a time-control of the tool can be performed.

In a further technically advantageous embodiment of the method, the type of the used tool is optically recognised by the detection system. This likewise provides e.g. the technical advantage that suitable control can be selected automatically on the basis of the recognised tool.

Exemplified embodiments of the invention are illustrated in the drawings and are described in more detail hereinunder.

In the figures:

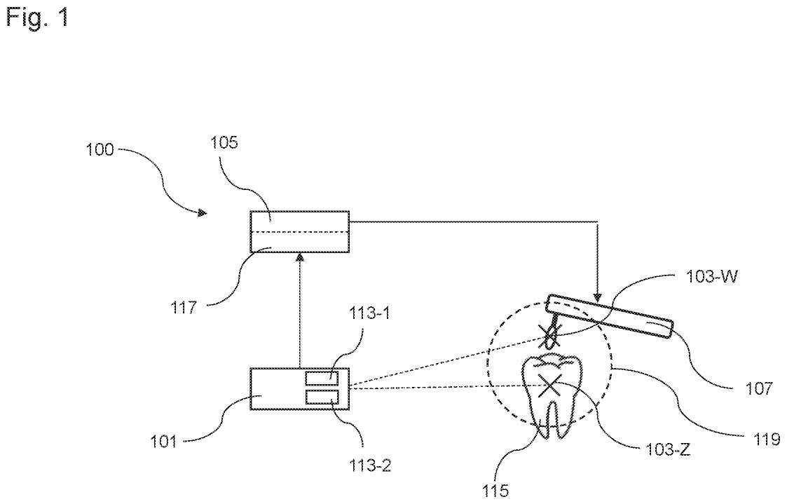

FIG. 1 shows a schematic view of a dental tool system;

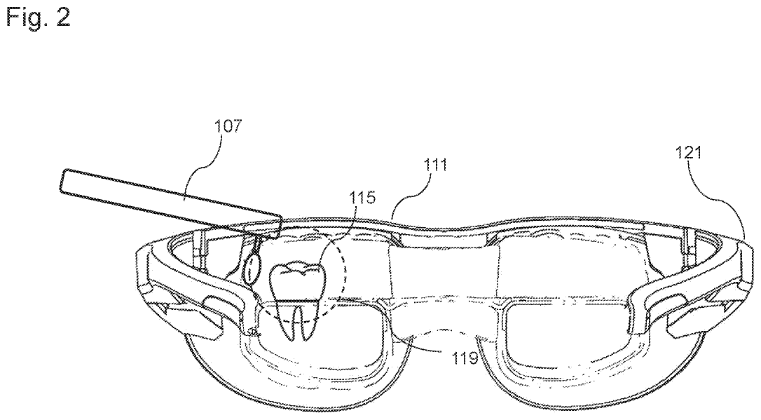

FIG. 2 shows data glasses for viewing for the dental tool system; and

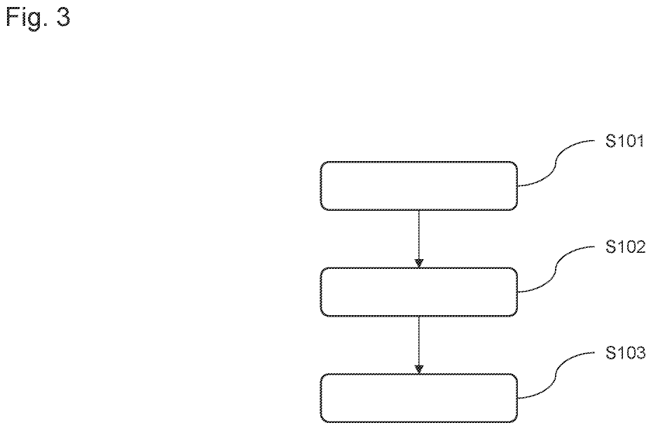

FIG. 3 shows a block diagram of a method for controlling a dental tool.

FIG. 1 shows a schematic view of a dental tool system 100. The dental tool system 100 serves to control or regulate a tool 107 which is used for a dental treatment. The tool 107 can be e.g. a polymerisation lamp, a dental drill, an endoscope, a camera, a scanner, a mirror, a light source for diagnostic purposes or a light source for colour determination. However, in general, the tool 107 can be any controllable tool which is used as part of a dental treatment. Controlling of the tool 107 is performed on the basis of a detected tool position 103-W and a position 103-Z of a tooth 115 or a tooth situation. The position 103-Z of the tooth 115 can be not only the position of a natural tooth but also the position of an artificial tooth, such as e.g. a bridge, a partial prosthesis, or even an implant or an abutment. The tooth situation is e.g. the region of a caries infestation or the spatial inner region of the tooth 115.

The tool position 103-W and the position 103-Z of the tooth 115 or the tooth situation can be detected by an optical detection system 101, an X-ray method, a magnetic resonance method or a computer tomography method.

The tool position 103-W or the position 103-Z can also be detected by means of radio labels (RFID chips) which are integrated in the tool 107 or the tooth 115. The tool position 103-W or the position 103-Z of the tooth 115 or the tooth situation can also be determined using a Kalman-filter which contributes to approximating the calculation of the actual position of the tool 107. The Kalman-filter as a mathematical method bridges missing measurement values or combines different data, such as optical data and data of an integrated, inertial measuring unit (IMU). However, in general, all methods are possible with which the tool position 103-W or the position 103-Z of the tooth 115 or the tooth situation can be detected.

The electronic detection system 101 generates e.g. a data record which includes or describes the position of the tooth 115 and also of the tool 107. The tool position 103-W and the position 103-Z of the tooth 115 or the tooth situation can be given by three-dimensional coordinates in any reference coordinate system. The position 103-Z of the tooth 115 is e.g. the centre of the tooth 115 and the tool position 103-W is the centre of a drill head. However, in general, the respective position can also be provided by other reference points, since it is easily possible to convert between the coordinate systems thus defined.

The electronic detection system 101 comprises e.g. a stereoscopic camera system comprising two cameras 113-1, 113-2. By stereoscopically recording pictures with the two cameras 113-1, 113-2, the tool position 103-W and the position 103-Z of the tooth 115 or the tooth situation can be easily detected and reconstructed. From the respective images of the cameras 113-1, 113-2 at different parallax angles or by means of image comparison, it is possible to calculate the tool position 103-W and the position 103-Z of the tooth 115 or the tooth situation by means of a computer algorithm.

The detection system 101 can also comprise a camera which detects individual distances using a travel time method of light (TOF (time of flight) camera). For this purpose, the oral cavity with the tooth 115 and the tool 107 is illuminated by means of a light pulse and the time required by the light to travel to the object and back again is measured for each image point. This time is directly proportional to the distance. The camera provides a spatial model and provides for each pixel the distance of the imaged tooth 115 and the tool 107 so that the respective positions of the tooth 115 and the tool 107 can be calculated.

The detection system 101 can detect the tool position 103-W and the position 103-Z of the tooth 115 or the tooth situation also based on a sequence of images of the tooth 115 which have been obtained from different viewing angles. For this purpose, a calculating method can be used which uses the individual images to reconstruct the tool position 103-W and the position 103-Z of the tooth 115 or tooth situation. To this end, in addition to the tool position 103-W and the position 103-Z of the tooth 115 or the tooth situation, information relating to the spatial position of the detection system 101 can be used.

The detected tool position 103-W and the position 103-Z of the tooth 115 or the tooth situation is input as a data record to an electronic calculating device 117. The calculating device 117 can detect e.g. whether the tool position 103-W is outside or inside a specified spatial region 119 with respect to the position 103-Z of the tooth 115 or the tooth situation. The spatial region 119 can be automatically set on the basis of the used tool 107. The spatial region 119 can be specified spherically around the position 103-Z of the tooth 115 or the tooth situation. In general, the spatial region 119 can also be in any other spatial shape. For example, the spatial region 119 can have different dimensions when a polymerisation lamp is used as the tool 107 than when a drill is used as the tool 107.

An electronic control device 105 controls the tool 107 on the basis of the detected tool position 103-W in relation to the position 103-Z of the tooth 115 or the tooth situation. A control scheme which is tailored to the tool 107 can be used for this purpose. In addition, a message can be output to a user of the dental tool system 100 if the tool 107 is located outside or inside the specified spatial region 119. Moreover, the user can be provided with haptic, acoustic or visual feedback relating to the tool 107.

The dental tool system 100 can be designed to generate an electronic file, such as e.g. a video file or CAD file, which documents a course of a treatment. This file can store the detected tool positions 103-W and the positions 103-Z of the tooth 115 or the tooth situation. As a result, information relating to the treatment can be permanently stored electronically so that the work performed on the tooth 115 can be tracked and examined retrospectively on the basis of the electronic file.

The electronic calculating device 117 and the control device 105 are each formed e.g. by a software module which is executed on a computer device having a processor and an electronic memory for storing the software module and the data records. In addition, the calculating device 117 or the control device 105 can be designed to calculate a tooth shape on the basis of detected optical images. The tooth shape is e.g. the actual spatial shape of a natural tooth, a bridge or a crown.

If the tool 107 is e.g. a polymerisation lamp for curing a filling compound, then it can be automatically switched on or off as soon as it is located within the specified spatial region 119 around the tooth 115. This can ensure that the polymerisation lamp illuminates the correct tooth 115. Moreover, safety of the treatment can be improved and an elevated temperature of the dental pulp caused by the polymerisation lamp is avoided. For this purpose, the tool position 103-W can be used to determine how long the tool 107 is located inside the spatial region 119, i.e. a time interval.

In contrast, if the tool 107 is a dental drill, then it can be activated only as soon as it is located in the specified spatial region 119 around the tooth 115. If the dental drill leaves the specified spatial region 119 around the tooth 115, it can be automatically switched off or a rotational speed can be reduced. As a result, injuries caused by the dental drill in the region of the oral cavity can be prevented.

If the tool 107 is a light-curing apparatus it is possible to detect whether it is being held at a suitable position in order to cure the material to be cured. The light-curing apparatus can be controlled or regulated on the basis of the detected tool position in relation to the position 103-Z of the tooth 115 by automatically switching on the light-curing apparatus as soon as the distance between it and the position 103-Z goes below a specified distance, or by automatically switching off the light-curing apparatus as soon as the distance between it and the position 103-Z is less than a specified distance. In the same manner, a time interval or light intensity of the illumination can be controlled. However, in the case of the light-curing apparatus, the light intensity, the wavelength or a polarisation degree of the emitted light can also be controlled in dependence on the detected tool position 103-W in relation to the position 103-Z. Compared with the tool state and the tooth or gum situation, the properties can be detected by measuring the wavelength and its effect on the surrounding area.

In the case of a dentist's chair as a dental tool 107, it can be moved and adjusted in dependence on the tool position in relation to the position 103-Z if it is too far away or light conditions are not appropriate. However, in general, other tools 107 can also be controlled in accordance with their properties on the basis of the detected tool position 103-W in relation to the position 103-Z.

The detection system 101 or the calculating device 117 can be designed to optically recognise the type of the used tool. The type indicates e.g. the specific type and the model of the used tool 107. For example, the type of tool 107 can be effected by reference to an image comparison with previously stored digital images, e.g. by means of neural networks. Otherwise, the tool 107 could also be provided with a miniaturised bar code or QR code by which the type of tool 107 can be recognised. In turn, the spatial region 119 for the tool 107 can be selected automatically in dependence on the recognised type of tool 107.

FIG. 2 shows data glasses 111 for viewing for the dental tool system 100. One or a plurality of optical recording devices of the electronic detection system 101 can be integrated into these data glasses 111. This increases the ability to handle the dental tool control system 100. In general, the detection system 101 can also be provided as a separate apparatus, by which the tool position 103-W and the position 103-Z of the tooth 115 or the tooth situation is detected.

The data glasses 111 (also called augmented-reality-glasses or smart glasses) are a wearable apparatus which is able to virtually project information in front of the eyes of the wearer of the glasses while said wearer is still able to visually perceive the environment. As a result, information can be displayed and added in the vision field of the wearer. For this purpose, the data glasses 111 comprise a display device 121 which can be formed by a display screen in close proximity to the eyes or a projector for directly projecting on the retina.

The data glasses 111 can additionally also comprise sensors for detecting movement of the head or for recognising the spatial position of the data glasses 111, such as e.g. a gyro sensor. However, the spatial position of the data glasses 111 can also be affected on the basis of specified optical reference points which are disposed in the environment of the data glasses 111, such as e.g. by means of trilateration or triangulation.

As a result, the spatial position of the data glasses 111 can be taken into consideration when detecting and calculating the tool position 103-W and the position 103-Z of the tooth 115 or the tooth situation so that the accuracy of the detected tool position 103-W and position 103-Z of the tooth 115 or the tooth situation is increased.

The data glasses 111 can additionally display the spatial region 119 for the tool 107. This spatial region 119 can be optically superimposed onto the visually perceived image e.g. by colour accentuation or a broken line. Furthermore, errors or warnings can be optically displayed on the data glasses 111 when using the tool 107.

FIG. 3 shows a block diagram of a method for controlling the dental tool. The method comprises the step S101 of optically detecting the spatial position 103-Z of the tooth 115 or the tooth situation and a spatial tool position 103-W of the tool 107. The spatial position 103-Z of the tooth 115 or the tooth situation and the spatial tool position 103-W can be detected continuously in real time. Subsequently, in step S102 the tool 107 is controlled on the basis of the detected tool position 103-W in relation to the position 103-Z of the tooth 115 or the tooth situation. As a result, errors in treatment can be avoided and the ability to handle tools 107 can be increased.

All features explained and illustrated in conjunction with individual embodiments of the invention can be provided in different combinations in the subject matter in accordance with the invention in order to achieve the advantageous effects thereof at the same time.

All the method steps can be implemented by devices which are suitable for carrying out the respective method step. All functions which are carried out by features relating to an apparatus can be a method step of a method.

The scope of protection of the present invention is defined by the claims and is not limited by the features explained in the description or shown in the figures.

### LIST OF REFERENCE SIGNS

100 dental tool system
101 detection system
103-Z position of the tooth or the tooth situation
103-W tool position
105 device for controlling or regulating
107 tool
109 recording device
111 data glasses
113 display device
115 tooth
117 calculating device
119 spatial region

1. A dental tool system, comprising:
a detection system (101) for detecting a spatial position (103-Z) of a tooth (115) or a tooth situation and a spatial tool position (103-W) of a tool (107); and
a device (105) for controlling or regulating the tool (107) on the basis of the detected tool position in relation to the position (103-Z) of the tooth (115) or the tooth situation.

2. The dental tool system (100) as claimed in claim 1,
wherein an optical recording device (109) of the electronic detection system (101) is integrated into data glasses (111).

3. The dental tool system (100) as claimed in claim 2,
wherein the dental tool system (100) is designed to optically display an error on a display device (113) of the data glasses (111).

4. The dental tool system (100) as claimed in claim 1,
wherein the detection system (101) comprises a stereoscopic detection system having a first and a second camera (113-1, 113-2).

5. The dental tool system (100) as claimed in claim 1,
wherein the detection system (101) comprises a camera for detecting the position (103-Z) of the tooth (115) or the tooth situation and the tool position (103-W) based upon a travel time measurement of light.

6. The dental tool system (100) as claimed in claim 1,
wherein the detection system (101) is designed to detect the position (103-Z) of the tooth (115) or the tooth situation and the tool position (103-W) based on a sequence of images.

7. The dental tool system (100) as claimed in claim 1,
wherein the electronic detection system (101) is designed to determine a time interval in which the tool position (103-W) is within a specified spatial region (119) with respect to the position (103-Z) of the tooth (115) or the tooth situation.

8. The dental tool system (100) as claimed in claim 1,
wherein the electronic detection system (101) is designed to optically recognise the type of tool (107) which is used.

9. The dental tool system (100) as claimed in claim 1,
wherein the tool (107) comprises a polymerisation lamp, a dental drill, an endoscope, a camera, a scanner, a mirror, a light-curing apparatus, a dentist's chair, a light source for diagnostic purposes or a light source for colour determination.

10. The dental tool system as claimed in claim 1,
wherein the detection system (111) comprises an autofocus function or zoom function for detecting the position (103-Z) of the tooth (115) or the tooth situation and/or tool position.

11. The dental tool system as claimed in claim 1,
wherein the detection system (111) is designed to detect the spatial position (103-Z) of the tooth (115) or the tooth situation based on a sequence of images of the tooth (115) which have been obtained from different viewing angles.

12. A method for controlling a dental tool, comprising the steps of:
detecting (S101) a spatial position (103-Z) of a tooth (105) or a tooth situation and a spatial tool position (103-W) of a tool (107); and
controlling or regulating the tool (107) on the basis of the detected tool position (103-W) in relation to the position 103-Z of the tooth (115) or the tooth situation.

13. The method as claimed in claim 12,
wherein an error is optically displayed on a display device (113) of data glasses (111).

14. The method as claimed in claim 12,
wherein the position (103-Z) of the tooth (115) or the tooth situation and the tool position (103-W) are detected by a stereoscopic detection system or by a detection system based on a travel time measurement of light.

15. The method as claimed in claim 12,
wherein the position (103-Z) of the tooth (115) or the tooth situation and the tool position (103-W) are detected based on a sequence of images.

16. The method as claimed in claim 12,
wherein a time interval is determined in which the tool position (103-W) is within a specified spatial region (119) with respect to the position (103-Z) of the tooth (115) or the tooth situation.

17. The method as claimed in claim 12,
wherein the type of tool (107) used is recognised optically or electronically by the detection system (101).
